(19) United States
(12) Patent Application Publication
(10) Pub. No.: US 2026/0018292 A1
(43) Pub. Date: Jan. 15, 2026

(54) METHODS AND SYSTEMS FOR ECG-DIAGNOSIS USING ZERO SHOT INFERENCE OF LARGE LANGUAGE MODELS

(71) Applicant: William Marsh Rice University, Houston, TX (US)

(72) Inventors: Akane Sano, Houston, TX (US); Han Yu, Houston, TX (US); Peikun Guo, Houston, TX (US)

(73) Assignee: William Marsh Rice University, Houston, TX (US)

(21) Appl. No.: 19/268,699

(22) Filed: Jul. 14, 2025

Related U.S. Application Data
(60) Provisional application No. 63/670,570, filed on Jul. 12, 2024.

(51) Int. Cl. G16H 50/20; A61B 5/346

(52) U.S. Cl. CPC G16H 50/20; A61B 5/346

(57) ABSTRACT

A method for diagnosing a health condition from electrocardiogram (ECG) data. The method may include obtaining the ECG data from an ECG machine and extracting a plurality of features from the ECG data resulting in raw extracted ECG features. The method may further include modifying the raw extracted ECG features to engineered ECG features based on a diagnosis guidance obtained from a database of domain knowledge using retrieval augmentation. The database of domain knowledge having been previously prepared and storing, at least, historical ECG data. The method may further include obtaining augmentation information from the database of domain knowledge using the engineered ECG features. The method may further include preparing a prompt that includes the engineered ECG features, the diagnosis guidance, and the augmentation information. The method may further include determining a health condition diagnosis based on the prompt using zero-shot inference with a large language model (LLM).

### CROSS REFERENCE TO RELATED APPLICATIONS

This application claims priority to U.S. provisional patent application No. 63/670,570, filed Jul. 12, 2024, which is herein incorporated by reference.

### STATEMENT REGARDING FEDERALLY SPONSORED RESEARCH

This invention was made with government support under Grant No. 2047296, awarded by the National Science Foundation.

### BACKGROUND

Electrocardiograms (ECGs) are a characterization of electrical activity within the heart and are routinely used to diagnose potential health conditions. Despite their ubiquity, accurately diagnosing health conditions from ECG data can be challenging. In particular, analysis of ECG data is typically performed by a trained expert with limited time. Moreover, due to the complexity of electrical activity often exhibited in the heart, there is a risk of misinterpretation and for multiple health conditions being present that are difficult to distinguish. Large language models (LLMs) are powerful machine learning models trained on vast data volumes and that may be capable of diagnosing health conditions rapidly. However, when applied to highly technical tasks, like diagnosing health conditions, LLMs may be biased depending on their training data or produce otherwise unreliable output. Accordingly, there exists a need to develop new methods and systems capable of leveraging LLMs to provide accurate and reliable diagnoses of health conditions, for example, from ECGs.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments disclosed herein relate to a method for diagnosing a health condition. The method includes obtaining observed electrocardiogram (ECG) data from an ECG machine. The method further includes extracting a plurality of features from the observed ECG data resulting in raw extracted ECG features and modifying the raw extracted ECG features, using retrieval augmentation and according to a diagnosis guidance obtained from a database of domain knowledge resulting in engineered ECG features. The database of domain knowledge having been constructed using, at least, historical ECG data. The method further includes obtaining augmentation information from the database of domain knowledge using, as a query, the engineered ECG features. The method yet further includes preparing a prompt including the engineered ECG features, the diagnosis information, and the augmentation information. The method yet further includes determining a health condition diagnosis based on the prompt using zero-shot inference with a large language model (LLM).

In one or more embodiments, preparing the prompt includes categorizing the engineered ECG features resulting in categorized ECG features. In such embodiments, the prompt may include at least some of the categorized ECG features. In one or more embodiments, the engineered features are categorized into general ECG information and lead-wise ECG information, where the lead-wise ECG information includes engineered ECG features specific to one or more leads of the engineered ECG data. In one or more embodiments, the prompt further includes formatting instructions relating to a format of the health condition.

In one or more embodiments, the diagnosis guidance is obtained by determining a subset of the raw extracted ECG features that are optimal for diagnosing a preselected health condition. In some embodiments, the engineered ECG features include the subset of the raw extracted ECG features determined by the diagnosis guidance.

In one or more embodiments, the method includes determining a plurality of health condition diagnosed, based, at least part, on the prompt using zero-short inference with the LLM.

In general, in one aspect, embodiments of the present disclosure relate to a system for diagnosing a health condition. The system includes an electrocardiogram (ECG) machine and a computer communicatively coupled to the ECG. The system further includes a database of domain knowledge relating to ECGs. The computer is configured to receive observed ECG data from the ECG machine. The computer is further configured to extract a plurality of features from the observed ECG data resulting in raw extracted ECG features and modify the raw extracted ECG features, using retrieval augmentation and according to a diagnosis guidance obtained from the database of domain knowledge, resulting in engineered ECG features. The computer is further configured to obtain augmentation information from the database of domain knowledge using, as a query, the engineered ECG features. The computer is further configured to prepare a prompt including the engineered ECG features, the diagnosis guidance, and the augmentation information. The computer is further configured to determine a health condition diagnosis based on the prompt using zero-shot inference with a large language model (LLM).

In one or more embodiments, the computer is configured to prepare the prompt based on the engineered ECG features. This preparation can include categorizing the engineered ECG features resulting in categorized ECG features. In such embodiments, the prompt may include at least some of the categorized ECG features. In one or more embodiments, the engineered features are categorized by the computer into general ECG information and lead-wise ECG information where the lead-wise ECG information includes engineered ECG features specific to one or more leads of the engineered ECG data.

In one or more embodiments, the computer is configured to obtain the diagnosis guidance by determining a subset of the raw extracted ECG features that are optimal for diagnosing a preselected health condition. In some embodiments, the engineered ECG features obtained with the computer include the subset of the raw extracted ECG features determined by the diagnosis guidance.

In one or more embodiments, the computer is configured to determine a plurality of health condition diagnoses, based, at least part, on the prompt using zero-short inference with the LLM.

In general, in one aspect, embodiments of the present disclosure relate to a non-transitory computer-readable medium storing instructions that, when executed by a processor, cause the processor to perform the following steps. The steps include obtaining observed electrocardiogram (ECG) data from an ECG. The steps further include extracting a plurality of features from the observed ECG data resulting in raw extracted ECG features and modifying the raw extracted ECG features, using retrieval augmentation and according to a diagnosis guidance obtained from the database of domain knowledge, resulting in engineered ECG features. The steps yet further include obtaining augmentation information from the database of domain knowledge using, as a query, the engineered ECG features. The steps yet further include preparing a prompt including the engineered ECG features, the diagnosis guidance, and the augmentation information. The steps yet further include determining a health condition diagnosis based on the prompt using zero-shot inference with a large language model (LLM).

In one or more embodiments, preparing the prompt, according to the instructions of the non-transitory computer-readable medium, includes categorizing the engineered ECG features resulting in categorized ECG features. In such embodiments, the prompt may include at least some of the categorized ECG features. In one or more embodiments, the engineered features are categorized, according to the instructions of the non-transitory computer-readable medium, into general ECG information and lead-wise ECG information, where the lead-wise ECG information comprises engineered ECG features specific to one or more leads of the engineered ECG data. In one or more embodiments, the prompt further includes formatting instructions relating to a format of the health condition.

In one or more embodiments, the diagnosis guidance is obtained, according to the instructions of the non-transitory computer-readable medium, by determining a subset of the raw extracted ECG features that are optimal for diagnosing a preselected health condition. In some embodiments, the engineered ECG features, according to the instructions of the non-transitory computer-readable medium, include the subset of the raw extracted ECG features determined by the diagnosis guidance.

In one or more embodiments, the instructions of the non-transitory computer-readable medium include determining a plurality of health condition diagnoses based, at least part, on the prompt using zero-short inference with the LLM.

### BRIEF DESCRIPTION OF DRAWINGS

FIG. 1 depicts an illustrative example of data that may be measured by an electrocardiogram machine.

FIG. 2 depicts a system in accordance with one or more embodiments.

FIG. 3 depicts a flowchart in accordance with one or more embodiments.

FIG. 4 depicts a neural network in accordance with one or more embodiments.

FIG. 5 depicts a transformer in accordance with one or more embodiments.

FIG. 6 depicts a system in accordance with one or more embodiments.

FIG. 7 depicts an overview of a system and method for zero-shot diagnosis of an electrocardiogram including an initial construction of a database for retrieval-augmentation in accordance with one or more embodiments.

FIG. 8 depicts a training process for a dual-modality model resulting in an electrocardiogram encoder that generates a textual description of an electrocardiogram in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

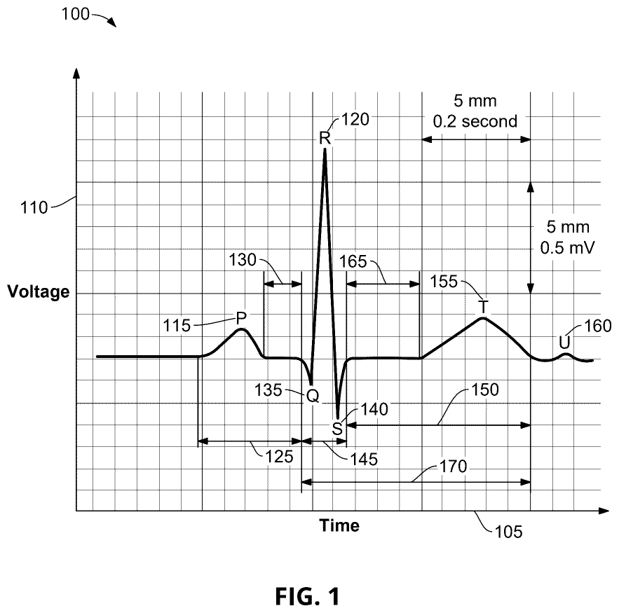

In the following description of FIGS. 1-8, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

It is to be understood that the singular forms “a,” “an,” and “the” include plural referents unless the context clearly dictates otherwise. Thus, for example, reference to “a signal” includes reference to one or more of such signals.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

Embodiments disclosed herein generally relate to an electrocardiogram (ECG) diagnosis method employing zero-shot retrieval-augmentation and large language models (LLMs). Conventionally, LLMs are pretrained using a vast corpora which can lead to the LLMs being biased toward some information (i.e., abundant information) or to hallucinate. This poses risks for many domain-specific tasks. For example, in an ECG diagnosis system it is essential to avoid such biased and misleading results. A potential fix is to acquire high-quality labeled (or annotated) ECG data for further training, or fine-tuning, LLMs, e.g., in a supervised manner. However, the acquisition of diagnostic labels for ECGs is usually expensive as they require the input of clinical professionals. Thus, developing an auto-diagnosis approach even without training samples can benefit a larger population.

The method disclosed herein makes use of at least one domain knowledge database, for example, including documents, textbooks, and papers relating to ECGs. Instead of depending on the existing knowledge of the LLMs, the process is augmented with steps of prompt preparation and answer generation by introducing expert domain knowledge to help the LLMs thoroughly understand the problem. Further, the feature selection and the prompt engineering processes are steered by the domain knowledge stored in the at least one domain knowledge database. Further, the final prompts by are augmented retrieving relevant documents of the observed ECG abnormalities for more accurate diagnosis.

Advantages of embodiments disclosed herein include more accurate inferences based on the introduction a retrieval-augmented ECG analysis model that integrates feature extraction and prompt design, both informed by domain expertise. Further, embodiments disclosed herein allow for zero-shot analysis of ECGs in relation to cardiac diseases as opposed to supervised models or few-shot tuned LLMs. Further, embodiments disclosed herein have demonstrated success in diagnosing arrhythmia and sleep apnea.

FIG. 1 shows example electrocardiogram ECG data (100). An electrocardiogram is a measure of the electrical activity, or voltage, in a heart as a function of time (i.e., an electrogram of the heart). As depicted in FIG. 1, the x-axis for the example ECG data (100) indicates time (105) while the y-axis indicates voltage (110). Electrocardiograms are measured using electrogram machines consisting of electrodes typically placed on a person's chest in proximity to the heart. Electrodes measure the difference in electrical potential between two locations. There are many possible configurations for electrodes in an ECG machine. For example, it is common for ECG machines to use ten electrodes placed across the body, with six electrodes placed on the chest and four placed on the limbs. However, alternative embodiments of ECG machines include devices such as a Holter monitor, which use variable number of electrodes (for example, two or five electrodes), or other wearable technology like smart watches.

Signals from electrodes may be combined, when multiple are available, to form different “leads” which measure the electrical activity of the heart according to a particular orientation angle. For example, in a ten electrode (twelve lead) ECG, it is common to define “Lead I” as the voltage between the positive left arm electrode and the right arm electrode. “Lead II” is commonly defined as the voltage between the positive left leg electrode and the right arm electrode. A person of ordinary skill in the art will understand that additional leads may be formed according to standard conventions or to other predefined conventions.

As depicted by the example ECG data (100), ECGs, measuring voltage (110) as a function of time (105), include a number of characteristic features. In the example ECG data (100) depicted in FIG. 1, a distance of one block along the x-axis (i.e., time (105)) indicates 0.2 seconds and may measure 5 mm extent. A distance of one block along the y-axis (i.e., voltage (110)), indicates 0.5 mV and also may measure 5 mm in extent. However, it is to be understood that FIG. 1 presents only an illustrative example that is not intended to be limiting to the present disclosure. In addition, A person of ordinary skill in the art will be familiar with the characteristic features of ECG data and so only a brief description is presented herein. Below includes a brief description of some of the characteristic features of ECG data.

The example ECG data (100) depicted in FIG. 1 includes a P-wave (115). Cells in the heart, at rest, are electrically polarized due different amounts of ion concentration on either side of cell membranes. Depolarization causes the cells to become less negatively charged and thus contract. The P-wave (115) represents atrial depolarization. The P-R interval (125) represents a passage of time between the onset of the P-wave (115) and the onset of the QRS complex. In contrast, the P-R segment (130) measures the time between the end of the P-wave (115) and the beginning of the Q-wave (135). It is common for voltage differences (along the y-axis, indicating voltage (110)), or the amplitude, to be measured with respect to the P-R segment (130). The QRS complex includes a Q-wave (135), an R-wave (120), and an S-wave (140) and represents the depolarization of the ventricles. The QRS interval (170) indicates the length of time spanned by the QRS complex. The Q-wave (135) represents the first negative deflection after the P-wave (115). The R-wave (120) represents the first positive deflection after the P-wave (115). The S-wave (140) represents the first negative deflection after the R-wave (120). After the QRS complex is the S-T segment (165), which represents the length of time between the end of the S-wave (140) and the start of the T-wave (155). The T-wave (155) represents the repolarization of the contractile cells. Following the T-wave (155) is a U-wave (160). The S-T interval (150) measures the time between the end of the S-wave (140) and the end of the T-wave (155). The Q-T interval (145) measures the length of the time between the start of the Q-wave and the end of the T-wave (155).

Generally, analysis of ECG data, such as example ECG data (100), is sometimes reduced to measuring characteristic features of the electrical signal as described above. A variety of health conditions can be diagnosed or indicated through analysis of the characteristic features of ECG data. A person of ordinary skill in the art will appreciate that additional characteristic features of ECG data may be present although they are not discussed or labeled in FIG. 1.

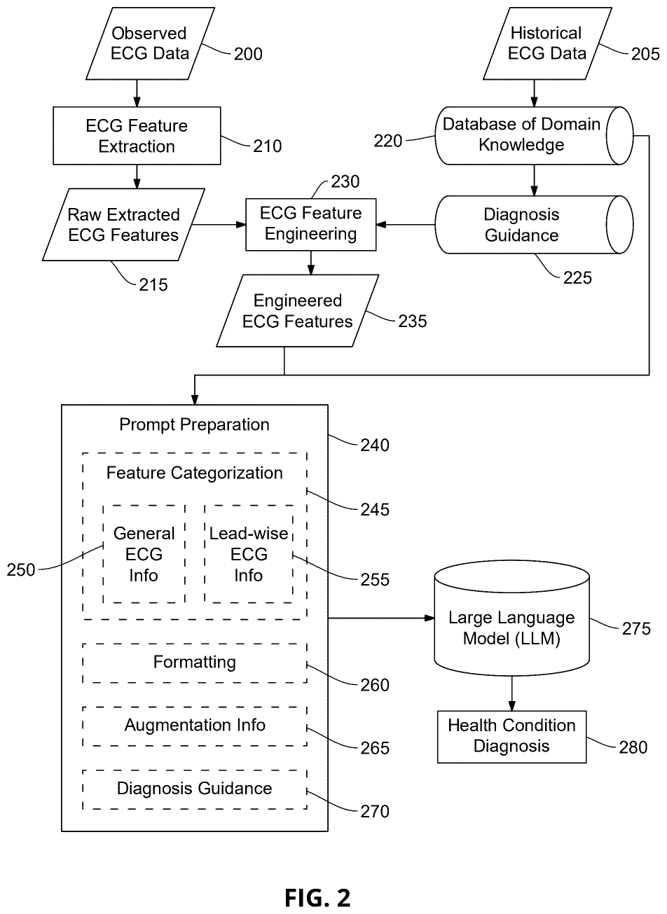

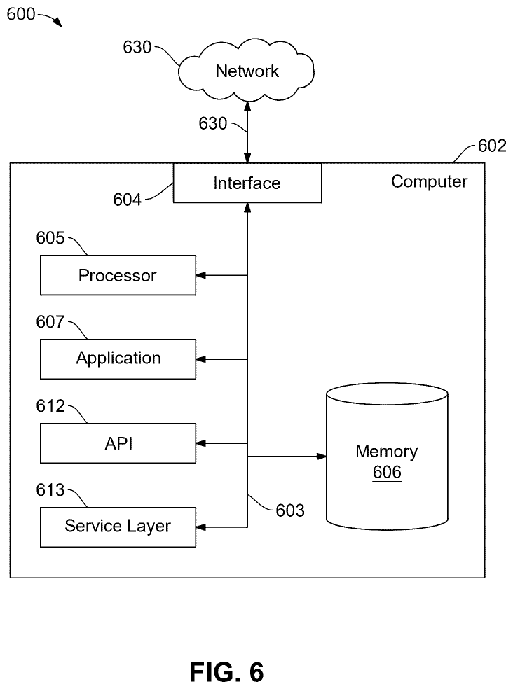

FIG. 2 represents a schematic diagram of a system for diagnosing a health condition in accordance with one or more embodiments. As depicted in FIG. 2, observed ECG data (200) is obtained. The observed ECG data (200) may be similar to the example ECG data (100) present in FIG. 1 and may be obtained by any ECG machine known in the art. ECG feature extraction (210) may be applied to the observed ECG data (200). As previously described, embodiments of the present disclosure may rely on utilizing a large language model (270), or LLM (270), to diagnose a health condition. A greater description of LLMs will be provided in reference to FIG. 6. Briefly, an LLM (270) is a type of machine learning model that has been trained on a vast volume of text in order to detect, measure, and reproduce the patterns of human text. Once trained, an LLM (270) is capable of interpreting input text in order to generate responses, summaries, and other types of content, such as answers to questions. As LLMs (270) are, by definition, trained to interpret text, they are not readily capable of interpreting observed ECG data (200) which is often numerical (e.g., represented by two vectors of numbers, one for time and one for amplitude). In order for the LLM (270) to be capable of interpreting or processing ECG data, such as observed ECG data (200), the observed ECG data (200) must be converted into text. ECG feature extraction (210) represents an initial conversion of the observed ECG data (200) to a text-based representation of the observed ECG data (200), resulting in raw extracted ECG features (215).

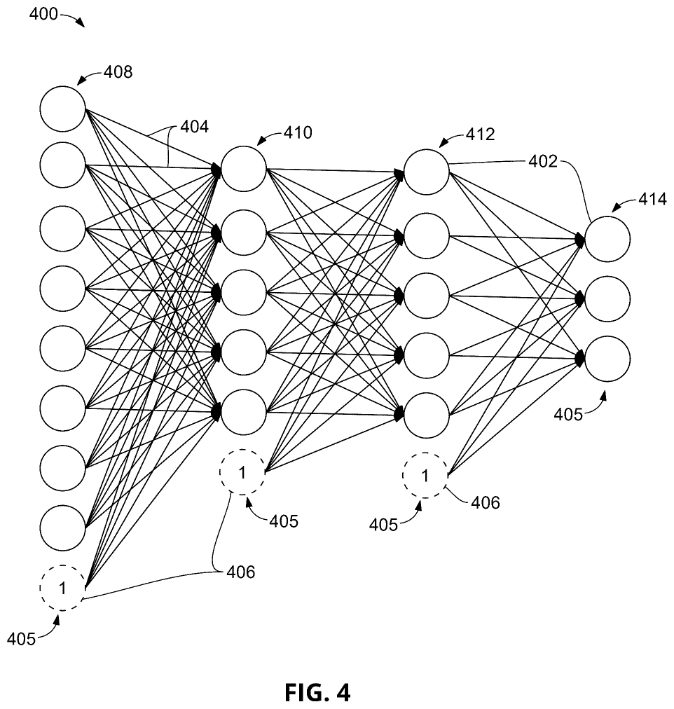

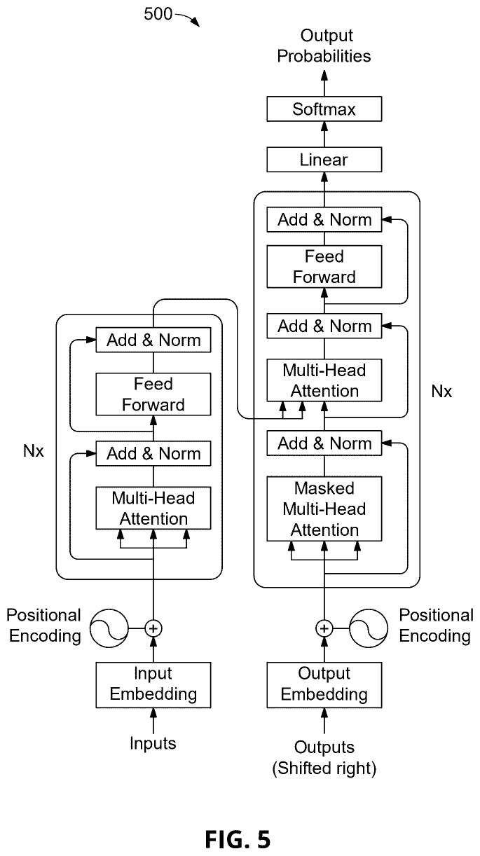

In one or more embodiments, extracting the plurality of features from the observed ECG data during ECG feature extraction (210) may include pretraining an ECG data encoder and using the pretrained ECG data encoder to extract features from the observed ECG data. An ECG data encoder is a system for converting the numerical information of ECG data into an embedding space that is interpretable by computers. Pretraining the ECG data encoder may include, for example, utilizing a training dataset in which standardized observed ECG data are paired with high-quality textual representations. In one or more embodiments, the high-quality textual representations may include demographic information about patients from which the observed ECG data was obtained, standard communication protocol (SCP) information, standard clinical labels, and machine generated reports obtained at the time of acquiring the observed ECG data. In addition, the high-quality textual representations may be further supplemented and modified by querying a database of domain knowledge using the methods of retrieval augmented generation (RAG) combined with large language models described below. Such methods may allow the combination of different ECG databases with different annotations and may provide further semantic context to the observed ECG data. The paired data (i.e., the observed ECG data and the text-based representation) are both encoded to an embedding space using data-type specific encoder models. For example, the ECG data encoder may be a convolutional neural network, while the text encoder may be a transformer model. Further information with respect to a neural network is given in reference to FIG. 4 below, while a general transformer model is described in FIG. 5. A contrastive loss function may be used to compare the embedded representations of the paired information in the embedding space. In addition, as one of the primary goals of ECG feature extraction (210) is to obtain a high-quality representation of the ECG data, additional learning criteria may be used. For example, the embedded representation of the numerical ECG data used in training may be decoded into text and compared with the original paired textual data using a captioning loss function. A captioning loss function that is similar to those used in image captioning tasks may be used, for example.

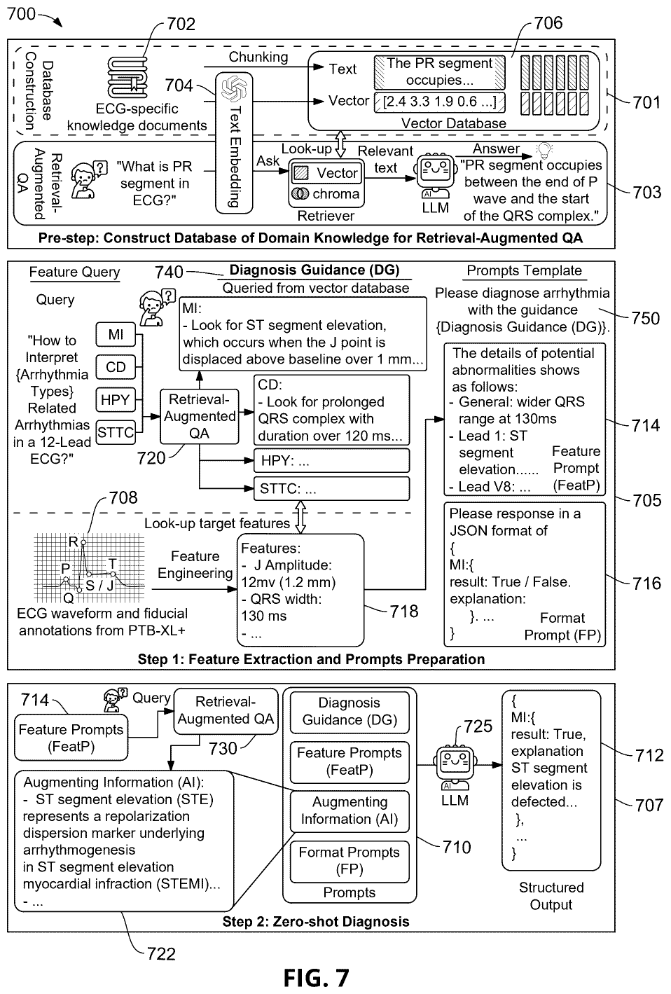

In one or more embodiments, a framework is established to construct a database and provide a text-based query mechanism (see Block 701 and Block 703 in FIG. 7). This framework can transform ECG condition labels into detailed descriptive text. The generated text incorporates demographic information, ECG conditions, and enriched waveform details. To leverage the enhanced interpretation of ECG conditions with domain expertise, a comprehensive vector database (e.g., database (706) of FIG. 7) can be constructed from domain-specific literature of authoritative medical texts. Examples of such literature include: Jane Huff, ECG workout: Exercises in arrhythmia interpretation. Lippincott Williams & Wilkins, 2006; and Tomas B Garcia, 12-lead ECG: The art of interpretation, Jones & Bartlett Learning, 2015. To extract and encode this information into a usable format, a text embedding can be employed (e.g., Open text-embedding-ada-002 provided by OpenAI). The resulting embeddings are then systematically organized, e.g., using the Chroma database management tool (see FIG. 7). In some embodiments, the embedding organization and database management are integrated with the LangChain Python library.

The above-described framework enriches the ECG-associated information through a comprehensive retrieval-augmented process. With the pre-constructed domain-knowledge database, the RAG-based approach enables the querying of related knowledge using given information such as standard clinical labels, standard communications protocol for computer-assisted ECG (SCP) statements, diagnostic interpretations, and machine-generated reports associated with ECG data. SCP statements are standardized textual formats that provide consistent documentation of ECG findings, following international protocols for computer-assisted ECG interpretation. While these datasets provide valuable diagnostic information, they often lack explicit details about the waveform patterns that are critical for a thorough ECG analysis. To address this gap, the database and querying framework described herein employs a RAG approach that enables it to query the knowledge database, as described, for relevant information and generate comprehensive textual descriptions of the potential waveform characteristics. For instance, SCP statements and standard arrhythmia diagnoses may not directly include detailed waveform descriptions; however, by querying the domain-knowledge database, enriched information can be synthesized, producing detailed descriptions of the waveform patterns that correspond to specific ECG conditions.

With the constructed RAG pipeline, waveform information can be queried using simple prompts such as “How is ECG Condition reflected in a 12-lead ECG?” The output can be organized utilizing an LLM, e.g., GPT-3.5, to generate the potential waveform details from the queried knowledge base. Take an example of the cardiac condition of the Right Bundle Branch Block (RBBB). By executing targeted queries in the database, descriptive context for specific waveform attributes can be generated and retrieved. For example, the ECG-associated information of “RBBB” is queried and converted into related waveform features, including “prolonged QRS duration” and “M-shaped RSR′ pattern in leads V1-V3.”

As discussed above, in some embodiments, the quality of representation extracted from ECG signals may be improved by pretraining a specialized ECG encoder alongside a textual encoder. In one or more embodiments, a dual-modality contrastive language-image pretraining (CLIP) is used to develop an ECG encoder and cross-modality decoder. In one or more embodiments, the ECG encoder is a one-dimensional modified version of the ConvNext v2 architecture (Sanghyun Woo, Shoubhik Debnath, Ronghang Hu, Xinlei Chen, Zhuang Liu, In So Kweon, and Saining Xie. Convnext v2: Co-designing and scaling convnets with masked autoencoders. In Proceedings of the IEEE/CVF Conference on Computer Vision and Pattern Recognition, pp. 16133-16142, 2023). This architecture is chosen considering the sequential nature of ECG waveform. In parallel, the textual encoder is developed. In one or more embodiments, the textual encoder utilizes BioLinkBERT, a derivative of the BERT architecture pretrained on biomedical texts, to effectively embed medical terminologies (Michihiro Yasunaga, Jure Leskovec, and Percy Liang. Linkbert: Pretraining language models with document links. arXiv preprint arXiv:2203.15827, 2022) BioLinkBERT is an extension of the standard BERT model (Jacob Devlin, Ming-Wei Chang, Kenton Lee, and Kristina Toutanova. Bert: Pre-training of deep bidirectional transformers for language understanding. arXiv preprint arXiv:1810.04805, 2018) specifically designed to improve the understanding of biomedical texts. Unlike traditional BERT, which processes each document independently, BioLinkBERT is pretrained on biomedical literature from PubMed, which takes advantage of the natural links between documents such as citations and references.

In accordance with one or more embodiments, two pretraining objectives for comprehensive learning are used: a contrastive loss for robust representation learning; and a captioning loss for semantic alignment. In the contrastive loss, the two encoders are jointly optimized by contrasting the paired text against others in the sampled batch:

$${L}_{c o n} = - \frac{1}{N} ( {\sum }_{i}^{N} log \frac{exp ( {S}_{i}^{T} \frac{{T}_{i}}{σ} )}{{\sum }_{j = 1}^{N} exp ( {S}_{i}^{T} \frac{{T}_{j}}{σ} )} + {\sum }_{i}^{N} log \frac{exp ( {T}_{i}^{T} \frac{{S}_{i}}{σ} )}{{\sum }_{j = 1}^{N} exp ( {T}_{i}^{T} \frac{{S}_{j}}{σ} )} ) ,$$

with Sᵢ and Tᵢ representing the normalized embeddings from the ECG signal and text encoders for the i-th ECG-text pair, N is the batch size during training, and σ as the temperature scaling factor. The first term in contrastive loss function, above, represents the ECG-to-text loss and the second term represents the text-to-ecg loss.

While the dual-encoder approach encodes the text as an embedding for the contrastive learning purpose, the generative approach aims for detailed granularity and requires the model to predict the exact tokenized texts with ECG and preceding texts. This approach encourages the encoders to capture the semantic information embedded in the texts actively. In one or more embodiments, the generated textual descriptions are aligned with the corresponding ECG signals by additionally defining a captioning loss L_{cap} similar to that used in image captioning tasks (e.g., Aaron van den Oord, Yazhe Li, and Oriol Vinyals. Representation learning with contrastive predictive coding. arXiv preprint arXiv:1807.03748, 2018):

$${L}_{c a p} = - {\sum }_{i}^{N} log ( P ( {t}_{i} \vert {t}_{< i} , {S}_{i} ; θ ) ) ,$$

where tᵢ represents the i-th token in the textual description, t_{<i} denotes all the preceding tokens, Sᵢ is the ECG signal, and θ represents the parameters of both encoders and the cross-modality decoder.

The overall pretraining objective is the combination of both contrastive loss and captioning loss, denoted as:

$$L = {λ}_{c o n} {L}_{c o n} + {λ}_{c a p} {L}_{c a p}$$

where λ_{con} and λ_{cap} are the loss weighting hyperparameters for the introduced objectives. In one or more embodiments, these two weighting parameters are each set to 1. By jointly optimizing these losses, a multimodal representation is learned that enriches the semantic link between ECG waveforms and their textual explanations. This method is anticipated to improve performances in downstream tasks that leverage the waveform details and demographics, such as diagnosing arrhythmia and performing large-scale patient identification using ECG data, as described below.

In one or more embodiments, a signal encoder is pretrained from scratch using three large-scale datasets with over 650,000 ECG-text training pairs. These datasets cover: Chapman-Shaoxing (Jianwei Zheng, Huimin Chu, Daniele Struppa, Jianming Zhang, Sir Magdi Yacoub, Hesham El-Askary, Anthony Chang, Louis Ehwerhemuepha, Islam Abudayyeh, Alexander Barrett, et al. Optimal multi-stage arrhythmia classification approach. Scientific reports, 10(1):2898, 2020); PTB-XL (Patrick Wagner, Nils Strodthoff, Ralf-Dieter Bousseljot, Dieter Kreiseler, Fatima I Lunze, Wojciech Samek, and Tobias Schaeffter. Ptb-xl, a large publicly available electrocardiography dataset. Scientific data, 7(1): 154, 2020); and MIMIC-ECG (Brian Gow, Tom Pollard, Larry A Nathanson, Alistair Johnson, Benjamin Moody, Chrystinne Fernandes, Nathaniel Greenbaum, Seth Berkowitz, Dana Moukheiber, Parastou Eslami, et al. Mimic-iv-ecg-diagnostic electrocardiogram matched subset. 2023). Each dataset contains 12-lead and 10-second ECG recordings sampled at 500 Hz. A detailed breakdown of each dataset is given as follows.

PTB-XL: This dataset consists of 21,837 12-lead, 10-second ECG recordings from 18,885 participants. In one or more embodiments, the training and test data split guidelines outlined in the original publication were followed such that encoder only used the training samples (17k) in the pretraining task. These samples include demographic data and SCP codes.

Chapman-Shaoxing: This dataset offers a larger set of 45k samples with associated demographic information and arrhythmia diagnoses.

MIMIC-IV-ECG: This dataset has 600k samples accompanied by demographics and machine-generated ECG reports.

The variety and volume of data provide a comprehensive foundation for the pretraining of models.

In one or more embodiments, the ECG signal encoder utilizes a 1D ConvNeXt-base backbone as the default architecture. This choice allows the model to effectively capture the spatial features within the ECG signal data. For text encoding, BioLinkBert is the default due to its proven capabilities in handling biomedical text data. In one or more embodiments, the AdamW optimizer is employed for optimization during the pretraining process with an initial learning rate of 5×10-5 to facilitate efficient convergence. To further adjust the learning rate throughout training, a warm-up phase of 5 epochs out of the total 30 epochs can be implemented. This warm-up phase allows the model to gradually adjust to the training data before applying the main learning rate. Additionally, in one or more embodiments, a learning rate decay of 0.1 is introduced after every 10 epochs to prevent overfitting in the later stages of training.

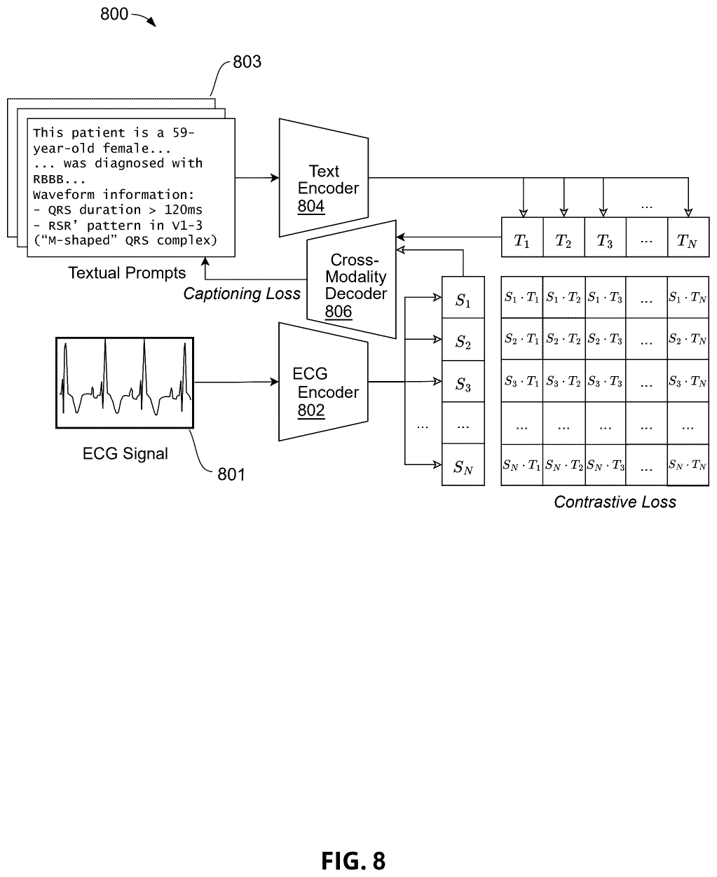

In accordance with one or more embodiments, the training process (800) to develop an ECG encoder capable of receiving an ECG and obtaining an textual description, or other features, is depicted in FIG. 8. As seen in FIG. 8, an ECG signals (801) are processed by the ECG encoder (802) to form signal embeddings, or vector representations of the ECG signals, S. Similarly, an associated, or paired, textual description (or prompt) (803) is processed by the text encoder (804) to form text embeddings, or vector representations of the text information, T. The contrastive loss is used to compare, using the above-listed equation, the signal and text embeddings to guide the parameterization of, at least, the ECG encoder (802). Further, a cross-modality decoder (806) is used, along with the captioning loss, to align generated textual descriptions with the corresponding ECG signals (801).

The above described ECG encoder and database can be used to infer information from ECG signals without any task-specific fine-tuning (e.g., see FIG. 7, Block 708 Block 718, Block 714). For example, ECG signal's embedding can be compared against a range of possible textual labels for different cardiac conditions without task-specific fine-tuning. The textual description that has the closest embedding distance to the ECG signal's embedding is selected. The closest distance can be determined using a similarity metric such as cosine similarity.

In review, in one or more embodiments, a multimodal contrastive pretraining framework is used to enhance the quality and robustness of representations learned from ECG signals. This framework integrates both contrastive and captioning capabilities to foster a deeper semantic understanding of ECG signals. Further, to address the lack of descriptive text associated with ECGs, a retrieval-augmented generation (RAG) pipeline is developed where this pipeline generates detailed textual descriptions for ECG data with demographic information, potential conditions, and waveform patterns.

Greater detail with respect to training a machine learning model, such as a neural network, is provided below in reference to FIG. 4. In short, pretraining the ECG data encoder entails iteratively determining an embedded representation of the training ECG data and an embedded representation of the paired text-based data. During each iteration, a comparison is made between the embedded representations of the ECG data, and the weights of the ECG data encoder are updated such that the embedded representations more closely agree. Generally, pretraining the ECG data encoder and using the pretrained ECG data encoder upon the observed ECG data (200) may result in more reliable and robust raw extracted ECG features (215).

The raw extracted ECG features (215) generally include a text-based representation of the observed ECG data (200). The raw extracted ECG features (215) may include any number of characteristic features of the observed ECG data (200), such as those described in relation to FIG. 1. For example, the raw extracted ECG features (215) may include a text-based representation of the P-wave (115), indicating its amplitude (in voltage) and duration (across time). As another example, the raw extracted ECG features (215) may describe, in words, the elevation or amplitude of the P-R segment. Any of the characteristic features of the observed ECG data (200) generically described in relation to FIG. 1 may be included in the raw extracted ECG features (215), in addition to characteristic features not described. That is, a person of ordinary skill in the art will appreciate that there are a number of ways that observed ECG data (200) may undergo ECG feature extraction (210) to obtain raw extracted ECG features (215) that represent the information of the observed ECG data (200) in a text-based format.

A common challenge when using LLMs (270) to address highly technical problems or questions, such as diagnosing health conditions, is that the LLM (270) may produce unreliable output. LLMs (270), by their very nature, are often trained on enormous volumes of text. For example, some LLMs (270) are trained on the “Common Crawl” dataset, which is a digital archive of approximately the entire internet spanning roughly 10 petabytes and billions of internet pages. Consequently, LLMs (270) can produce medically inaccurate responses because the majority of the training data is not vetted or provided by trained medical professionals. LLMs (270) may fabricate answers when a truly definitive answer cannot be found or provide generic responses to a specific question. Additionally, LLMs (270) are sensitive to the construction of the input data, or prompt, consisting of the text to be interpreted. For example, prompts that are too long or contain too much information may be difficult for the LLM (270) to interpret. One option to overcome these limitations of LLMs (270) is to perform retrieval-augmented generation feature selection (sometimes referred to as retrieval augmented generation, or RAG).

Retrieval augmentation, in accordance with one or more embodiments, includes constructing a database of domain knowledge (220) using historical ECG data (205) to obtain a diagnosis guidance (225). Depending on the medical application, the observable presentation, or symptoms, of a particular health condition may be known. For example, as ECGs have been used extensively in medical settings to analyze heart conditions, there are patterns, waveforms, and characteristic features present ECGs that are known to be correlated with one or more health conditions. Consequently, retrieval augmentation may be used to query an external, and medically reliable, dataset that provides information regarding the characteristics of ECGs associated with particular health conditions. In one or more embodiments, a database of domain knowledge (220) is constructed using historical ECG data (205). The historical ECG data (205) may include textbooks, publications, journal articles, and other resources that provide medically accurate information. The database of domain knowledge (220) may be constructed via text embedding of the historical ECG data (205), whereby alphabetical text is converted into numerical vectors. As a consequence of the retrieval augmentation, a diagnosis guidance (225) may be obtained. The diagnosis guidance (225) describes the particular characteristic features that may be present in ECG data that are most strongly associated or correlated with one or more health conditions. In one or more embodiments, the diagnosis guidance is obtained by determining a subset of the raw extracted ECG features that are optimal for diagnosing a preselected health condition.

In accordance with one or more embodiments, the diagnosis guidance (225) is used during ECG feature engineering (230) involving the raw extracted ECG features (215) to obtain engineered ECG features (235). As previously described, the raw extracted ECG features (215) may include a variety of text-based descriptions of the observed ECG data (200). However, a significant portion of the information that may be provided by the raw extracted ECG features (215) may not be useful for diagnosis, and including all of the information may present difficulties for the LLM (270). The diagnosis guidance (225) may be used during ECG feature engineering (230) to reduce the raw extracted ECG features (215) to those that are most useful for diagnosing a preselected health condition. Consequently, the engineered ECG features (235) may include the subset of the raw extracted ECG features determined by the diagnosis guidance (225) to be optimal for diagnosing a preselect health condition. The engineered ECG features (235) may also include a written description, according to the diagnosis guidance (225), indicating which particular features of an ECG are most relevant for diagnosing one or more health conditions.

A number of additional aspects of feature engineering may be included in ECG feature engineering (230). For example, ECG feature engineering (230) may also include data pre-processing. Pre-processing includes applying one or more transformations of the data, such processing via a mathematical function, as data cleaning or removal of inconsistent measurements or outliers, normalization, filtering, convolution, and other techniques not mentioned. Data pre-processing techniques that rely on numerical methods are applicable as long as they numerical information is returned to a text-based representation. Thus, the raw extracted ECG features (215) may be modified, using retrieval augmentation and according to a diagnosis guidance (225) obtained from the database of domain knowledge (220), resulting in engineered ECG features (235).

In one or more embodiments, the engineered ECG features (235) are used in prompt preparation (240) to prepare a prompt. A prompt is the final input to the LLM (270) and represents the text to be interpreted. As described previously, the output of an LLM (270) is sensitive to the structure and content of the prompt. In one or more embodiments, prompt preparation (240) includes feature categorization (245), where the engineered ECG features are categorized according to the information of the observed ECG data (200) that they represent. For example, the engineered features may be categorized into general ECG information (250) and lead-wise ECG information (255). The lead-wise ECG information (255) includes a subset of the engineered ECG features (235) that is specific to one or more leads of engineered ECG features (235). The general ECG information (250) includes information that provides a global description of the engineered ECG features (235). For example, the general ECG information (250) may include information that is averaged or summed across all of the leads present in the engineered ECG features (235). Both the general ECG information (250) and the lead-wise ECG information (255) may include a description of abnormal features that are identified in the engineered ECG features (235). Such abnormalities may be indicative of one or more health conditions. Separating information specific to particular leads within the lead-wise ECG information (255) may provide greater detail reveal regarding particular abnormalities associated with one or more health conditions.

In one or more embodiments, prompt preparation (240) includes formatting (260). Formatting (260) involves specifying the structure the structure of both the input prompt as well as the output of the LLM (270). For example, formatting (260) may involve structuring the prompts such that they present the categorized features uniformly. Formatting (260) the input prompt ensures that information, as interpreted by the LLM (270) is not biased. Formatting (260) as applied to the output of the LLM (270) involves requesting, as part of the prompt, that the output of the LLM (270) be presented in a specific format (i.e., formatting instructions for the response). For example, the output of the LLM (270) could be formatted in easily interpretable prose such that a patient could understand it. In one or more embodiments, formatting (260) of the LLM (270) output includes requesting the output to be presented in a predetermined JSON format.

In one or more embodiments, prompt preparation (240) further includes forming a prompt with a prompt preface (not shown in FIG. 2, but shown in Examples given below). The prompt preface can introduce the task to be performed by the LLM including identification of the included data, such as the diagnosis information (225), extracted (or engineered) features (235), augmentation information (265), and how it should be used.

In one or more embodiments, prompt preparation includes obtaining augmentation information (265) which is added to the prompt. The augmentation information (265) is used to provide medically reliable context for the engineered ECG features (235) that are reported in the prompt. Similar to obtaining the diagnosis guidance (225), the augmentation information (265) may be obtained by querying the database of domain knowledge (220) to determine how to interpret abnormalities in the engineered ECG features (235). Abnormalities, in this context, refer to deviations from standard ECG measurements, or heartbeat patterns, that may be associated with one or more health conditions. For example, the engineered ECG features (235) may indicate an S-T segment elevation. Then, by querying database of domain knowledge, augmentation information (265) may be obtained including a medically reliable interpretation of S-T segment elevation, such as a description of how S-T segment elevation is correlated with myocardial injury.

In accordance with one or more embodiments, the LLM (270) is used to determine a health condition diagnosis (275) based, at least in part, on the prompt, using zero-shot inference. Zero-shot inference refers to obtaining an output from the LLM (270) without any specific training of the LLM (270) or fine-tuning. As has been briefly described above (and will be described in greater detail with respect to FIG. 5), LLMs (270) are commonly trained on enormous volumes of text. However, LLMs (270) may be further trained for application in a specific area or fine-tuned. However, application-specific training and fine-tuning require extensive resources in terms of medical data, human-involvement, and computational resources. Thus, methods and systems capable of providing reliable health condition diagnoses (275) without the use of application-specific training and fine tuning of LLMs (270) are desirable. Accordingly, the prompt preparation (240) described by the present disclosure is used, in accordance with one or more embodiments, to allow successful and reliable zero-shot inference using the LLM (270) to obtain a health condition diagnosis (275). The health condition diagnosis (275) may include a binary classification (e.g., “True” or “False”) indicating whether a health condition is identified. The health condition diagnosis (275) may also include a probability of whether a health condition is identified. The health condition diagnosis (275) may also include a written description explaining why the health condition diagnosis (275) was given.

In one or more embodiments, a plurality of health condition diagnoses (275) is determined, based, at least in part, on the prompt using zero-short inference with the LLM (270). The plurality of health condition diagnoses (275) may describe multiple health conditions being present in a single individual specified by the prompt. Alternatively, the plurality of health condition diagnoses (275) may describe the health conditions of many individuals specified by the prompt.

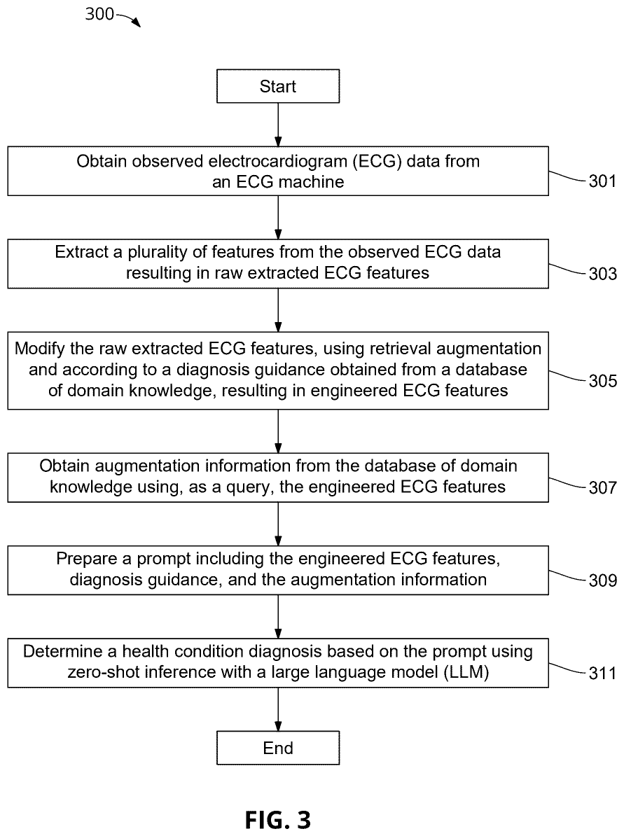

FIG. 3 depicts a flowchart for diagnosing a health condition in accordance with one or more embodiments of the present disclosure. In Block 301, observed electrocardiogram (ECG) data may be obtained from an ECG machine. It is noted that steps of the flowchart of FIG. 3 rely on a previously constructed database of domain knowledge. The database of domain knowledge may be constructed, for example, using historical ECG data as previously described. In Block 305, a plurality of features from the observed ECG data may be extracted, resulting in raw extracted ECG features. In one or more embodiments, extracting the plurality of features from the observed ECG data may include pretraining an ECG data encoder and using the pretrained ECG data encoder to extract features from the observed ECG data. In Block 307, the raw extracted ECG features may be modified using retrieval augmentation and according to a diagnosis guidance obtained from the database of domain knowledge, resulting in engineered ECG features. In Block 308, augmentation information may be obtained from the database of domain knowledge using, as a query, the engineered ECG features. That is, retrieval augmentation is used to expand, or focus, the knowledge base of the LLM to data or information with relevance to the ECG begin diagnosed. Relevant textual information on the ECG being diagnosed is obtained as augmentation information by querying the potential ECG abnormalities. Differing from the diagnosis guidance, the augmentation information is queried based on extracted (or engineered features) described during prompt preparation (see feature categorization (245) in FIG. 2), e.g., ST segment elevation and prolonged QRS complex. This step aims to retrieve information derived from specific features so that provides a more detailed context for these abnormalities.

In Block 309, a prompt may be prepared. The prompt can include the engineered ECG features, the diagnosis guidance, and the augmentation information. The prompt can further include formatting instructions, e.g., for how the output or response of the LLM should be formed. The prompt can further include a prompt preface, e.g., selected from a template, that indicates how the LLM should use the information contained in the prompt. In Block 311, a health condition diagnosis may be determined based on the prompt using zero-shot inference with the LLM.

FIG. 7, described later in the instant disclosure, depicts an overview of a system and method, e.g., following the steps of the flowchart of FIG. 3, for zero-shot diagnosis of a ECG. FIG. 7 further depicts the construction of the database for retrieval-augmentation in accordance with one or more embodiments.

In accordance with one or more embodiments, embodiments of the present disclosure may make use of artificial neural networks (“neural networks”). FIG. 4 depicts a neural network in accordance with one or more embodiments. At a high level, a neural network (400) may be graphically depicted as being composed of nodes (402), where here any circle represents a node, and edges (404), shown here as directed lines. The nodes (402) may be grouped to form layers (405). FIG. 1 displays four layers (408, 410, 412, 414) of nodes (402) where the nodes (402) are grouped into columns, however, the grouping need not be as shown in FIG. 1. The edges (404) connect the nodes (402). Edges (404) may connect, or not connect, to any node(s) (402) regardless of which layer (405) the node(s) (402) is in. That is, the nodes (402) may be sparsely and residually connected. A neural network (400) will have at least two layers (405), where the first layer (408) is considered the “input layer” and the last layer (414) is the “output layer.” Any intermediate layer (410, 412) is usually described as a “hidden layer”. A neural network (400) may have zero or more hidden layers (410, 412) and a neural network (400) with at least one hidden layer (410, 412) may be described as a “deep” neural network or as a “deep learning method.” In general, a neural network (400) may have more than one node (402) in the output layer (414). In this case the neural network (400) may be referred to as a “multi-target” or “multi-output” network.

Nodes (402) and edges (404) carry additional associations. Namely, every edge is associated with a numerical value. The edge numerical values, or even the edges (404) themselves, are often referred to as “weights” or “parameters.” While training a neural network (400), numerical values are assigned to each edge (404). Additionally, every node (402) is associated with a numerical variable and an activation function. Activation functions are not limited to any functional class, but traditionally follow the form

$$A = f ( {\sum }_{i ∈ ( i n comi ng )} [ {( node value )}_{i} {( edge value )}_{i} ] )$$

where i is an index that spans the set of “incoming” nodes (402) and edges (404) and ƒ is a user-defined function. Incoming nodes (402) are those that, when viewed as a graph (as in FIG. 1), have directed arrows that point to the node (402) where the numerical value is being computed. Some functions for ƒ may include the linear function ƒ(x)=x, sigmoid function

$$f ( x ) = \frac{1}{1 + {e}^{- x}} ,$$

an rectified linear unit function ƒ(x)=max(0, x), however, many additional functions are commonly employed. Every node (402) in a neural network (400) may have a different associated activation function. Often, as a shorthand, activation functions are described by the function ƒ by which it is composed. That is, an activation function composed of a linear function ƒ may simply be referred to as a linear activation function without undue ambiguity.

When the neural network (400) receives an input, the input is propagated through the network according to the activation functions and incoming node (402) values and edge (404) values to compute a value for each node (402). That is, the numerical value for each node (402) may change for each received input. Occasionally, nodes (402) are assigned fixed numerical values, such as the value of 1, that are not affected by the input or altered according to edge (404) values and activation functions. Fixed nodes (402) are often referred to as “biases” or “bias nodes” (406), displayed in FIG. 1 with a dashed circle.

In some implementations, the neural network (400) may contain specialized layers (405), such as a normalization layer, or additional connection procedures, like concatenation. One skilled in the art will appreciate that these alterations do not exceed the scope of this disclosure.

As noted, the training procedure for the neural network (400) comprises assigning values to the edges (404). To begin training the edges (404) are assigned initial values. These values may be assigned randomly, assigned according to a prescribed distribution, assigned manually, or by some other assignment mechanism. Once edge (404) values have been initialized, the neural network (400) may act as a function, such that it may receive inputs and produce an output. As such, at least one input is propagated through the neural network (400) to produce an output. Recall, that a given data set will be composed of inputs and associated target(s), where the target(s) represent the “ground truth,” or the otherwise desired output.

The neural network (400) output is compared to the associated input data target(s). The comparison of the neural network (400) output to the target(s) is typically performed by a so-called “loss function;” although other names for this comparison function such as “error function,” “misfit function,” and “cost function” are commonly employed. Many types of loss functions are available, such as the mean-squared-error function, however, the general characteristic of a loss function is that the loss function provides a numerical evaluation of the similarity between the neural network (400) output and the associated target(s). The loss function may also be constructed to impose additional constraints on the values assumed by the edges (404), for example, by adding a penalty term, which may be physics-based, or a regularization term. Generally, the goal of a training procedure is to alter the edge (404) values to promote similarity between the neural network (400) output and associated target(s) over the data set. Thus, the loss function is used to guide changes made to the edge (404) values, typically through a process called “backpropagation.”

While a full review of the backpropagation process exceeds the scope of this disclosure, a brief summary is provided. Backpropagation consists of computing the gradient of the loss function over the edge (404) values. The gradient indicates the direction of change in the edge (404) values that results in the greatest change to the loss function. Because the gradient is local to the current edge (404) values, the edge (404) values are typically updated by a “step” in the direction indicated by the gradient. The step size is often referred to as the “learning rate” and need not remain fixed during the training process. Additionally, the step size and direction may be informed by previously seen edge (404) values or previously computed gradients. Such methods for determining the step direction are usually referred to as “momentum” based methods.

Once the edge (404) values have been updated, or altered from their initial values, through a backpropagation step, the neural network (400) will likely produce different outputs. Thus, the procedure of propagating at least one input through the neural network (400), comparing the neural network (400) output with the associated target(s) with a loss function, computing the gradient of the loss function with respect to the edge (404) values, and updating the edge (404) values with a step guided by the gradient, is repeated until a termination criterion is reached. Common termination criteria are: reaching a fixed number of edge (404) updates, otherwise known as an iteration counter; a diminishing learning rate; noting no appreciable change in the loss function between iterations; reaching a specified performance metric as evaluated on the data or a separate hold-out data set. Once the termination criterion is satisfied, and the edge (404) values are no longer intended to be altered, the neural network (400) is said to be “trained”.

Modern day large language models (LLMs) typically make use of transformer network architectures (500), which are a particular type of machine learning model. FIG. 5 depicts a schematic diagram of a transformer network architecture (500) in accordance with one or more embodiments. A full description of transformer network architectures is beyond the scope of this disclosure. However, to promote a basic understanding, a brief description is presented herein.

As has been discussed, LLMs, are used to interpret and respond to text or written words. An LLM utilizing a transformer network architecture (500) begins by receiving a text-based input. Generally, transformers include an input embedding layer that converts the input text into a numerical vector. More specifically, sequences of texts are “tokenized” by an encoder, which breaks a sequence of text into individual components called tokens. Transformers seek to accomplish “next-token prediction” in which given a sequence of tokens, the subsequent token is generated. Positional encoding is used to characterize the positional information of tokens in a sequence. Thus, both the meaning of the word itself as well as its position is characterized during encoding. Transformers utilize a multi-head attention layer to describe the importance of each token in the sequence and the relative importance of preceding and subsequent tokens. Residual connections and normalization layers are used to stabilize the output of the multi-head attention layer. A feed-forward layer, or a small neural network may be used to process tokens at each respective position in the sequence. The feed-forward layer is typically used to process output from one multi-head attention layer such that it is better prepared for another multi-head attention layer. The output of the encoder, which is the encoded text that has been processed to determine the information of each token and their mutual positional relevance, is used as the input for a decoder layer. A decoder layer includes many of the same features as the encoder layer, including embedding, multi-head attention layers, and normalization layers. A predetermined “start of sequence” token is typically used to begin the output, and the following tokens are determined by the decoder according to the above-described mechanisms. A linear transformation layer may be used to act as a classifier on the output of the decoder, which projects the high-dimensional output (e.g., a tensor) of the decoder into a lower-dimensional (e.g., a vector) classification which is often a collection of words in the vocabulary recognized by the transformer. Finally, a softmax layer may be used to assign probabilities to each element (or class) determined by the linear layer. The element, or word (or class) with the highest probability is selected as the next element in the sequence. These steps repeat until a predetermined “end of sequence” token is determined.

A person of ordinary skill in the art will appreciate that many modifications may be made to the transformer network architecture (500) described above.

Embodiments disclosed herein may be implemented on a computer system. FIG. 6 is a block diagram of a computer system (602) used to provide computational functionalities associated with described algorithms, methods, functions, processes, flows, and procedures as described in the instant disclosure, according to one or more embodiments. The illustrated computer (602) is intended to encompass any computing device such as a server, desktop computer, laptop/notebook computer, wireless data port, smart phone, personal data assistant (PDA), tablet computing device, one or more processors within these devices, or any other suitable processing device such as an edge computing device, including both physical or virtual instances (or both) of the computing device. An edge computing device is a dedicated computing device that is, typically, physically adjacent to the process or control with which it interacts.

Additionally, the computer (602) may include a computer that includes an input device, such as a keypad, keyboard, touch screen, or other device that may accept user information, and an output device that conveys information associated with the operation of the computer (602), including digital data, visual, or audio information (or a combination of information), or a GUI.

The computer (602) may serve in a role as a client, network component, a server, a database or other persistency, or any other component (or a combination of roles) of a computer system for performing the subject matter described in the instant disclosure. In some implementations, one or more components of the computer (602) may be configured to operate within environments, including cloud-computing-based, local, global, or other environment (or a combination of environments).

At a high level, the computer (602) is an electronic computing device operable to receive, transmit, process, store, or manage data and information associated with the described subject matter. According to some implementations, the computer (602) may also include or be communicably coupled with an application server, e-mail server, web server, caching server, streaming data server, business intelligence (BI) server, or other server (or a combination of servers).

The computer (602) may receive requests over network (630) from a client application (for example, executing on another computer (602) and responding to the received requests by processing the said requests in an appropriate software application. In addition, requests may also be sent to the computer (602) from internal users (for example, from a command console or by other appropriate access method), external or third-parties, other automated applications, as well as any other appropriate entities, individuals, systems, or computers.

Each of the components of the computer (602) may communicate using a system bus (603). In some implementations, any or all of the components of the computer (602), both hardware or software (or a combination of hardware and software), may interface with each other or the interface (604) (or a combination of both) over the system bus (603) using an application programming interface (API) (612) or a service layer (613) (or a combination of the API (612) and service layer (613). The API (612) may include specifications for routines, data structures, and object classes. The API (612) may be either computer-language independent or dependent and refer to a complete interface, a single function, or even a set of APIs. The service layer (613) provides software services to the computer (602) or other components (whether or not illustrated) that are communicably coupled to the computer (602). The functionality of the computer (602) may be accessible for all service consumers using this service layer. Software services, such as those provided by the service layer (613), provide reusable, defined business functionalities through a defined interface. For example, the interface may be software written in JAVA, C++, or other suitable language providing data in extensible markup language (XML) format or another suitable format. While illustrated as an integrated component of the computer (602), alternative implementations may illustrate the API (612) or the service layer (613) as stand-alone components in relation to other components of the computer (602) or other components (whether or not illustrated) that are communicably coupled to the computer (602). Moreover, any or all parts of the API (612) or the service layer (613) may be implemented as child or sub-modules of another software module, enterprise application, or hardware module without departing from the scope of this disclosure.

The computer (602) includes an interface (604). Although illustrated as a single interface (604) in FIG. 6, two or more interfaces (604) may be used according to particular needs, desires, or particular implementations of the computer (602). The interface (604) is used by the computer (602) for communicating with other systems in a distributed environment that are connected to the network (630). Generally, the interface (604) includes logic encoded in software or hardware (or a combination of software and hardware) and operable to communicate with the network (630). More specifically, the interface (604) may include software supporting one or more communication protocols associated with communications such that the network (630) or interface's hardware is operable to communicate physical signals within and outside of the illustrated computer (602).

The computer (602) includes at least one computer processor (605). Although illustrated as a single computer processor (605) in FIG. 6, two or more processors may be used according to particular needs, desires, or particular implementations of the computer (602). Generally, the computer processor (605) executes instructions and manipulates data to perform the operations of the computer (602) and any algorithms, methods, functions, processes, flows, and procedures as described in the instant disclosure.

The computer (602) also includes a memory (606) that holds data for the computer (602) or other components (or a combination of both) that may be connected to the network (630). The memory may be a non-transitory computer readable medium. For example, memory (606) may be a database storing data consistent with this disclosure. Although illustrated as a single memory (606) in FIG. 6, two or more memories may be used according to particular needs, desires, or particular implementations of the computer (602) and the described functionality. While memory (606) is illustrated as an integral component of the computer (602), in alternative implementations, memory (606) may be external to the computer (602).

The application (607) is an algorithmic software engine providing functionality according to particular needs, desires, or particular implementations of the computer (602), particularly with respect to functionality described in this disclosure. For example, application (607) may serve as one or more components, modules, applications, etc. Further, although illustrated as a single application (607), the application (607) may be implemented as multiple applications (607) on the computer (602). In addition, although illustrated as integral to the computer (602), in alternative implementations, the application (607) may be external to the computer (602).

There may be any number of computers (602) associated with, or external to, a computer system containing computer (602), wherein each computer (602) communicates over network (630). Further, the term “client,” “user,” and other appropriate terminology may be used interchangeably as appropriate without departing from the scope of this disclosure. Moreover, this disclosure contemplates that many users may use one computer (602), or that one user may use multiple computers (602).

FIG. 7 depicts an overview of a system and method for zero-shot diagnosis of a ECG including the construction of the database for retrieval-augmentation in accordance with one or more embodiments. In particular, FIG. 7 is discussed in the context of a zero-shot diagnosis method and system diagnosing arrhythmia and sleep apnea using ECG data.

As shown in Block 701, a database (706) of domain knowledge is constructed. In accordance with one or more embodiments, the database (706) is a vector database. The database (706) is constructed by storing documents (702) related to ECGs. In the case that the database (706) is a vector database, as shown in FIG. 7, the documents (702) can be vectorized using a text embedding (704).

In the present example, for the use of ECG in arrhythmia diagnosis the database (706) can include guidance from two published books: (1) ECG Workout: Exercises In Arrhythmia Interpretation by Huff (Jane Huff; ECG workout: Exercises in arrhythmia interpretation. Lippincott Williams & Wilkins, (2006)) and (2) 12-Lead ECG: The Art of Interpretation by Garcia (Tomas B Garcia; 12-lead ECG: The art of interpretation. Jones & Bartlett Learning, (2015)). Similarly, for diagnosing sleep apnea the database (706) can store an encoding of a apnea-related textbook and various papers (e.g.: Winfried J Randerath, Bernd M Sanner, and Virend K Somers. Sleep apnea: current diagnosis and treatment, volume 35. Karger Medical and Scientific Publishers, 2006; Laiali Almazaydeh, Khaled Elleithy, and Miad Faezipour. Detection of obstructive sleep apnea through ecg signal features. In 2012 IEEE International Conference on Electro/Information Technology, pages 1-6. IEEE, 2012.; M Drinnan, J Allen, P Langley, and A Murray. Detection of sleep apnea from frequency analysis of heart rate variability. In Computers in Cardiology 2000. Vol. 27 (Cat. 00CH37163), pages 259-262. IEEE, 2000.; J N McNames and A M Fraser. Obstructive sleep apnea classification based on spectrogram patterns in the electrocardiogram. In Computers in Cardiology 2000. vol. 27 (Cat. 00CH37163), pages 749-752. IEEE, 2000.; C W Zywietz, V Von Einem, B Widiger, and G Joseph. Ecg analysis for sleep apnea detection. Methods of information in medicine, 43(01):56-59, 2004.).

In one or more embodiments, the text embedding (704) is text-embedding-ada-002 embedding extraction API (OpenAI, 2023). Further, in one or more embodiments, the extracted embedding is managed using the Chroma database tool in conjunction with the LangChain Python library. This setup facilitates the search and retrieval of related text from the embedding space with appropriate prompts. Block 703 depicts this process, where a query is embedded into the embedding space and then used to identify relevant text that can be used to augment a prompt used by a LLM.

Block 705 depicts the process of feature extraction and prompts preparation. Prompts are crucial for guiding LLMs to generate relevant responses, especially for models that are not further fine-tuned (frozen LLMs). To transform an ECG, such as an observed or input ECG (708), into effective prompts, features must be engineered and extracted from the ECG. For example, for arrhythmia diagnosis, comprehensive features can include detailed fiducial information, where this information can be formed using commercial and open-source algorithms. Similarly, for sleep apnea diagnosis, a Python library like NeuroKit2 can be used to detect the fiducial points and extract features such as heart rate variability and spectral power.

Block 705 also depicts retrieval-augmented feature selection (720). While extracting features from original ECGs typically involves universal elements such as waveforms and amplitudes of fiducial points and intervals, the large number of diverse features across ECG leads presents a challenge. However, overloading LLMs with an extensive array of comprehensive features for reasoning and inference might not only exceed input length restrictions of LLMs but also may introduce redundant information, which potentially hinders accurate diagnosis. To mitigate this, feature extraction is refined by looking up domain specific databases and extracting crucial insights. This process includes querying targeted questions pertaining to the interpretation of specific arrhythmia types, such as ST/T segment change (STTC), myocardial infarction (MI), conduction disturbance (CD), and hypertrophy (HYP) (see FIG. 7, 720, for querying targeted questions). This method enables the identification of the most relevant features for each diagnostic category (see FIG. 7, 740, “Diagnosis Guidance”), which provides LLMs with clinically related data and helps avoid information overload.

Consequently, features are extracted (718) with the queried diagnosis guidance (740). In one or more embodiments, the extracted features (718) include 15 different fiducial points and segments across different leads such as QRS complex, T wave, P wave, PR segment, RS segment, etc. For example, J-point amplitude for ST-segment elevation and depression and the ratio of R/S amplitude for the waveform of RS complexes, etc. The same procedures are applied for the querying guidance (740) of diagnosing sleep apnea. For example, the extracted features can cover the average heart rate, variability of R-R intervals, elevation of spectral power in the VLF band, power in both the low-frequency (LF) and high-frequency (HF) bands, as well as the ratio of power between LF and HF bands.

Continuing with Block 705, a prompt can be prepared; or, at least, partially prepared. As seen, the prompt can include a prompt preface (750) that instructs the LLM what do with the rest of the information provided in the prompt. The prompt preface (750) can follow a fixed template. The prompt can further include the Diagnosis Guidance (740). As such, the prompt integrates the insights previously queried from the database (706), which cover the essential information on interpreting specific arrhythmia types or sleep apnea detection. The prompt can further include the extracted features (718) organized as a feature prompt (714). This incorporates detailed ECG information highlighting potential abnormalities that are converted from the extracted features. In accordance with one or more embodiments, this information is organized into two main categories including general information and leadwise information. General information covers general insights into the ECG, such as the QRS duration, providing an overview of the ECG and anomalies. Lead-wise information is included because abnormalities can present differently across the leads (e.g., 12 leads) of an ECG. As such, the lead-wise features integrate specific information for each lead, such as the waveform of the P and T waves. This ensures that the LLMs can discern and diagnose conditions that might be prominent in one lead but subtle or absent in other leads. Further, the prompt can include a format prompt or formatting instructions (716). The formatting instructions (716) can guide the LLMs to produce structured responses for easy post-processing. In one or more embodiments, the formatting instructions (716) instruct the LLMs to respond in a structured JSON format with each arrhythmia type as the first layer key followed by Boolean diagnosis results and reasoning explanation.

Block 707 depicts the zero-shot generation of a diagnosis, or response (712) (or output). In Block 707, the feature prompt (714) is used in another retrieval-augmentation process (730) to query the database (706) for ECG information relevant to the observed ECG (708). That is, Block 707 includes an initial step where the detailed ECG information prompts (e.g., feature prompt (714)) is used as a querying mechanism. Relevant textual information on the observed ECG (708) is retrieved (730) and labeled, or added to the prompt, as the augmenting information (722). Differing from the diagnosis guidance (see FIG. 7, 720), the augmenting information (722) is queried (see FIG. 7, 730) based on the features extracted in prompts (e.g., 714), e.g., ST segment elevation and prolonged QRS complex. This step aims to retrieve information derived from specific features as to provide a more detailed context for these abnormalities. For example, by querying keywords of “ST segment elevation”, the augmenting information (722) covers “This can be indicative of myocardial injury or infarction (heart attack). However, it can also be caused by other conditions such as coronary artery spasm, acute pericarditis, ventricular aneurysm, early repolarization pattern, hyperkalemia, or hypothermia . . . .” As seen in Block 707, the augmenting information (722) is added to the prompt, or forms a final prompt (710).

The final prompt (710) is executed by one or more LLMs (725) to produce a diagnosis or response (712). As such, the disclosed processes leverage LLMs to directly understand and infer the prompts without training or fine-tuning. In essence, embodiments disclosed herein ensure that the LLMs are consistently augmented with domain-specific insights, guaranteeing that the outputs are precise and reflect a deep-rooted understanding of the ECG condition and diagnosis.

Although the disclosure has been described with respect to only a limited number of embodiments, those skilled in the art, having benefit of this disclosure will appreciate that various other embodiments may be devised without departing from the scope of the present invention. Accordingly, the scope of the invention should be limited only by the attached claims.

### Results

The zero-shot retrieval-augmented system and method of this disclosure were applied to the diagnosis of arrhythmia, using the PTB-XL(+) datasets (see: Patrick Wagner, Nils Strodthoff, Ralf-Dieter Bousseljot, Dieter Kreiseler, Fatima I Lunze, Wojciech Samek, and Tobias Schaeffter. Ptb-xl, a large publicly available electrocardiography dataset. Scientific data, 7(1):154, 2020.; and Nils Strodthoff, Temesgen Mehari, Claudia Nagel, Philip J Aston, Ashish Sundar, Claus Graff, Jorgen K Kanters, Wilhelm Haverkamp, Olaf Dossel, Axel Loewe, et al. Ptb-xl+, a comprehensive electrocardiographic feature dataset. Scientific Data, 10(1):279, 2023.), and to the diagnosis of sleep apnea, using the Apnea-ECG dataset (see Thomas Penzel, George B Moody, Roger G Mark, Ary L Goldberger, and J Hermann Peter. The apnea-ecg database. In Computers in Cardiology 2000. Vol. 27 (Cat. 00CH37163), pages 255-258. IEEE, 2000.). Existing methods were also applied to diagnose arrhythmia and sleep apnea in these datasets for comparison to the zero-shot retrieval-augmented system and method of this disclosure. These existing methods include a supervised method and a method with few-shot tunning. As will be shown, zero-shot retrieval-augmented system and method demonstrates improved performance over existing methods, even when no training samples were used, highlighting its applicability in scenarios where labeled data is scarce or expensive to obtain. Specifically, the zero-shot retrieval-augmented system and method of this disclosure outperformed the few-shot LLM-based approach of a prior study and even achieved competitive results on fully trained supervised learning methods.

For evaluation of the zero-shot retrieval-augmented system and method of this disclosure open-source model and other methods that make use of LLMs, various LLMs were used. These include open-source models like LLaMA2 and the closed-source GPT-3.5 models.

LLaMA2: LLaMA2 is an LLM developed by Meta AI. LLaMA2 has 7 billion to 70 billion parameters, and it can be used for a variety of tasks, such as dialogue and question-answering. It has been shown to outperform other open-source LLMs on many benchmarks. LLaMA2 is available for free for research and commercial use. Due to the constraints on the computational resources, the 7B and 13B versions are employed herein.

GPT-3.5: GPT-3.5 is an LLM developed by OpenAI. GPT-3.5 has 175 billion parameters, which makes it one of the largest LLMs ever created. It can be used for a variety of tasks. It has been shown to outperform other LLMs on many benchmarks. Generally, GPT-3.5 is accessible via API calls.

The PTB-XL dataset is a large dataset containing 21,837 clinical 12-lead ECG records from 18,885 patients of 10-second length, where 52% are male and 48% are female with ages ranging from 0 to 95 years (median 62 and interquartile range of 22). There are two sampling rates: 100 and 500 Hz, available in the dataset. The raw ECG data are annotated by two cardiologists into five major categories, including normal ECG (NORM), myocardial infarction (MI), ST/T Change (STTC), Conduction Disturbance (CD), and Hypertrophy (HYP). The PTB-XL+ dataset covers algorithm-extracted features on the ECG sequences, such as durations, amplitudes, on/off-sets of segments, fiducial points, median beats, etc. The datasets contain a comprehensive collection of many different co-occurring pathologies and a large proportion of healthy control samples. To ensure a fair comparison of machine learning algorithms trained on the dataset, the recommended splits of training and test sets were followed. However, given that method and system disclosed herein employs a zero-shot approach, the training samples are not used to fine-tune the models.

The Apnea-ECG dataset contains 70 records of ECG recorded at a sampling rate of 100 Hz without features extracted, 35 of which are used for training and 35 for testing. The duration of the records ranges from almost 7 hours to nearly 10 hours. Labels indicating the presence or absence of sleep apnea are assigned to each minute of the recordings. Here, the ECG recordings are segmented into one-minute intervals, which results in 6000 data points for each segment. There are 17233 training samples and 17010 samples for the test set with a non-apnea to apnea sample ratio of 61.49% to 38.51%.

As stated, to evaluate the zero-shot retrieval-augmented system and method of this disclosure, the system and method is compared to a traditional supervised model and a state-of-the-art model that uses LLMs. The comparison models are described in greater detail as follows.

### Supervised Method

A 1D-CNN model is used as the supervised baseline. This model follows prior studies (e.g., Nils Strodthoff, Temesgen Mehari, Claudia Nagel, Philip J Aston, Ashish Sundar, Claus Graff, Jorgen K Kanters, Wilhelm Haverkamp, Olaf Dossel, Axel Loewe, et al. Ptb-xl+, a comprehensive electrocardiographic feature dataset. Scientific Data, 10(1):279, 2023). The 1D-CNN kernel is designed to capture the temporal patterns in the ECG sequences, making it suitable for tasks that require understanding the sequential nature of the ECGs. With the supervised baseline, a full training strategy is implemented, which leverages all the available training samples to help the model learn useful parameters from scratch.

Numerical Prompts with Few-Shot Tuning

Further, a method from a prior LLM-based study (see Hang Zhang, Xin Li, a Lidong Bing. Videollama: An instruction-tuned audio-visual language model for video understanding. arXiv preprint arXiv:2306.02858, 2023.) is used as a part of the evaluation for performance comparisons. This method converts ECG signals into a textual sequence of inter-beat-intervals (IBIs), e.g., “Identify the average heart rate from given interbeat interval sequence 896,1192,592,1024,1072,808,888 . . . ”, which shows promising results in detecting heart rates and Sinus rhythms in a 25-shot training setting. Due to the discrepancy between tasks of detecting heart rhythms and detecting cardiac diseases, the approach of the prior LLM-based study is reproduced by converting the ECGs into sequences of IBI numbers and enhancing the prompts by covering the location and amplitudes of fiducial points including P, T, Q, R, and S for each lead. Also, randomly sampled 25 ECGs are used as the training sample following the few-shot learning scheme. LoRA (Edward J Hu, Yelong Shen, Phillip Wallis, Zeyuan Allen-Zhu, Yuanzhi Li, Shean Wang, Lu Wang, and Weizhu Chen. Lora: Low-rank adaptation of large language models. arXiv preprint arXiv:2106.09685, 2021.), which is an efficient fine-tuning method widely applied for LLMs, is used in fitting the training data.

### Evaluation Results: Arrhythmia Diagnosis

Table I shows the evaluation performances on the arrhythmia diagnosis. The performances are evaluated in metrics of accuracy rate, macro precision, macro recall, and macro f1 score across all the classes. The GPT-3.5 model outperformed the open-source LLaMA2 models in all metrics. When comparing the zero-shot retrieval-augmented system and method of this disclosure with the supervised learning method, superior performances in accuracy rate, macro precision, and macro F1 scores are observed for the system and method of this disclosure; whereas the supervised method shows a higher macro recall score. This result suggests that zero-shot retrieval-augmented system and method of this disclosure can be effective in detecting arrhythmia even without leveraging any training samples.

TABLE I
The evaluation results of arrhythmia diagnosis in metrics of accuracy
rate, macro precision, macro recall, and macro F1 score.

| | | | | Macro | Macro | Macro |
|---|---|---|---|---|---|---|
| Method | Model | Training | Accuracy | Precision | Recall | F1 |
| Supervised | 1D-CNN | 17441 | 0.748 | 0.708 | 0.643 | 0.660 |
| Few-shot LLM | LLaMA2-7B | 25 | 0.417 | 0.391 | 0.277 | 0.357 |
| (Li et al., 2023) | LLaMA2-13B | 25 | 0.422 | 0.401 | 0.294 | 0.348 |
| Zero-shot RAG | LLaMA2-7B | 0 | 0.714 | 0.765 | 0.548 | 0.617 |
| (this disclosure) | LLaMA2-13B | 0 | 0.726 | 0.770 | 0.561 | 0.622 |
| | GPT-3.5 | 0 | 0.757 | 0.791 | 0.616 | 0.669 |

Few-shot LLM: few-shot textual numeric prompts. Zero-shot RAG: zero-shot retrieval-augmented generation. Bold represents the highest performances in the evaluation set.

The class-wise diagnostic performance offers insights into the efficacy and potential limitations of our zero-shot retrieval-augmented approach using the GPT-3.5 model. A deeper dive into the results, as presented in Table II, reveals patterns in diagnosis across various classes of arrhythmia. CD, HYP, and MI detection show high precision scores, indicating that once these conditions are detected, the false detecting rate remains relatively low. While precision is promising in certain classes, there have been instances where conditions were not detected and were instead misclassified as normal ECGs. This could be caused by the fact that the engineered features and prompts might not have captured comprehensive nuances associated with certain arrhythmia types. In addition, the detecting performance for STTC is relatively lower compared to the other arrhythmia classes.

The explanations generated by the LLM also provide some insights into our error analysis. For all samples incorrectly identified as HYP by LLMs, the explanations cite that the ECG matches the Sokolov-Lyon criteria for diagnosing HYP by checking the R waves in lead V1/V2 and S waves in lead V5/V6, even when HYP was not identified in the human annotated labels. Such inconsistencies might stem from information loss during the signal filtering process or flawed fiducial point annotations. The errors from signal processing can directly affect the precision of prompts. On the other hand, currently detecting STTC majorly depends on abnormalities observed in the T wave and the duration of PT. Another challenge arises when trying to precisely describe complex waveform patterns in textual data, such as the varying waveform morphology in real ECGs.

TABLE II
Class-wise performances for the zero-shot retrieval-
augmented method and system of this disclosure.

| Class | Samples | Precision | Recall | F1 Score |
|---|---|---|---|---|
| NORM | 912 | 0.54 | 0.79 | 0.61 |
| CD | 473 | 0.93 | 0.61 | 0.77 |
| HYP | 243 | 0.91 | 0.55 | 0.70 |
| MI | 415 | 0.80 | 0.63 | 0.70 |
| STTC | 516 | 0.77 | 0.50 | 0.58 |

### Evaluation Results: Sleep Apnea Diagnosis

Table III displays the performance of the examined method in diagnosing sleep apnea. This table reveals that the supervised learning method excels in terms of accuracy and precision scores. In contrast, the zero-shot retrieval-augmented method and system of thus disclosure using the GPT-3.5 model delivers the highest recall and F1 scores. Similar to the findings in arrhythmia diagnosis, the numeric prompts with the few-shot tuning method yield less-than-ideal results for the apnea task.

TABLE III
The evaluation results of sleep apnea diagnosis in metrics
of accuracy rate, precision, recall, and F1 score.

| Method | Model | Training | Accuracy | Precision | Recall | F1 |
|---|---|---|---|---|---|---|
| Supervised | 1D-CNN | 17233 | 0.821 | 0.804 | 0.843 | 0.787 |
| Few-shot LLM | LLaMA2-7B | 25 | 0.675 | 0.492 | 0.535 | 0.504 |
| (Li et al., 2023) | LLaMA2-13B | 25 | 0.691 | 0.512 | 0.562 | 0.522 |
| Zero-shot RAG | LLaMA2-7B | 0 | 0.753 | 0.710 | 0.855 | 0.758 |
| (this disclosure) | LLaMA2-13B | 0 | 0.772 | 0.728 | 0.859 | 0.770 |
| | GPT-3.5 | 0 | 0.804 | 0.763 | 0.910 | 0.801 |

Few-shot LLM: few-shot textual numeric prompts. Zero-shot RAG: zero-shot retrieval-augmented generation. Bold represents the highest performances in the evaluation set.

Despite the zero-shot retrieval-augmented method and system of this disclosure showing promise in recall rates, the LLM-based approaches produce a comparatively low precision score when compared with the supervised learning method. This disparity may arise from signal quality and prompt engineering precision. The prompts, engineered from features crafted based on R-R intervals extracted by software, are susceptible to signal noises. By combining the error analysis with signal quality check (see Zhidong Zhao and Yefei Zhang. Sqi quality evaluation mechanism of single-lead ecg signal based on simple heuristic fusion and fuzzy comprehensive evaluation. Frontiers in physiology, 9:727, 2018.), it is found that the average precision scores on test sequences in “excellent” quality (6.38% of all test sequences) are 6.4% higher than ECGs in “barely acceptable” quality (74.21% of all test sequences). Additionally, ECG processing software can mis-detect R peaks in some sequences, resulting in extended intervals that manifest as confusing features, even when the original signal is normal. Among the false-positive samples that are detected as ECG with apnea, 72.1% highlights either high VLF power or significant heart rate variability.

Finally, an ablation study is performed to assess the contribution of each component of the zero-shot retrieval-augmented method and system of this disclosure. The input prompt for the zero-shot retrieval-augmented method and system of this disclosure consists of diagnosis guidance, feature prompts, augmenting information, and format prompts, which build a comprehensive understanding of ECG signals and diagnostic information. Among these prompt components, the feature and format prompts are not removable as they function essentially in describing the ECGs and generating processable output, respectively. Thus, the performance of removing diagnosis guidance or augmenting information in prompts on the PTBXL+ dataset is evaluated to understand the impacts of these components.

Table IV shows the performances of removing specific components in prompts on the PTB-XL+ dataset with GPT-3.5. From the table, it is seen that when the Diagnosis Guidance (DG) component is removed, the F1 score drops from 0.669 to 0.593, indicating a decrease of 0.076. Removing the Augmenting Information component results in a smaller decrease in the F1 score from 0.669 to 0.628. When both the DG and Augmenting Information components are removed, the F1 score drops significantly to 0.571, which is a decrease of 0.098 from the full method. This suggests that both components contribute to the overall performance, with the DG component being more critical in the model performance than the Augmenting Information component.

TABLE IV
The evaluation results of sleep apnea diagnosis in metrics
of accuracy rate, precision, recall, and F1 score. Few-
shot LLM: few-shot textual numeric prompts. Zero-shot RAG:
zero-shot retrieval-augmented generation. Bold represents
the highest performances in the evaluation set.

| Removed Prompts | F1 | Diff. |
|---|---|---|
| None (Full Method) | 0.669 | — |
| Diagnosis Guidance (DG) | 0.593 | 0.076 |
| Augmenting Information | 0.628 | 0.041 |
| DG & AI | 0.571 | 0.098 |

These results underscore the potential and limitations of leveraging advanced language models, such as LLaMA2 and GPT-3.5, for complex medical diagnostic tasks such as arrhythmia and sleep apnea detection. The zero-shot retrieval-augmented method and system of this disclosure demonstrated promising performances, even when no training samples were used, highlighting its applicability in scenarios where labeled data is scarce or expensive to obtain. Further, the zero-shot retrieval-augmented method and system of this disclosure outperformed the few-shot LLM-based approach in a prior study and even achieved competitive results on fully trained supervised learning methods. While the efficacy of the zero-shot retrieval-augmented generation was showcased using ECG data, its potential extends further.

The zero-shot retrieval-augmented method and system of this disclosure can be effectively applied to an array of physiological signals such as photoplethysmogram (PPG) and electrodermal activity (EDA). Furthermore, this methodology can be adapted into a multimodal system to tackle more intricate diagnostic tasks and insights

### EXAMPLES

Two examples of using a LLM to diagnose an ECG are provided. In each example, the components of the prompt, namely, the prompt preface, the diagnosis guidance, the engineered ECG features, the augmenting information, and the formatting instructions are shown. Further, the resulting response produced by the LLM, having executed the prompt, is shown.

### Example 1: Normal ECG in PTB-XL+

### Prompt:

Preface: Identify the types of arrhythmia in the ECG signal with diagnostic guidance and the extracted features. The diagnostic guidance you should follow is detailed below. Additionally, consider the supplemental information from textbooks regarding the detected features. Please be careful about the features in the different leads.

Diagnosis Guidance: When diagnosing a Myocardial Infarction (MI), various ECG changes must be considered. The ST segment elevation is a critical indicator that signals myocardial injury. For instance, if the ST elevation is observed in leads II, III, and aVF, an inferior MI is suggested, whereas ST elevation in leads V2 to V4 points to an anterior MI. Additionally, ST depressions opposite the infarct area, known as reciprocal changes, are also significant. T wave abnormalities are another aspect, where inverted or sharply peaked “tombstone” T waves can be seen in the affected leads. Lastly, the presence of Q waves, which are pathological, indicates a transmural MI and will appear in the corresponding leads of the infarct area. Conduction disturbances in the heart manifest through various changes in the ECG. A QRS complex that is wider than 0.12 seconds is indicative of a disturbance. Specifically, an RSR′ or rSR′ pattern in lead V1 suggests a right bundle branch block (RBBB), whereas a wide S wave or notched R wave in lead V6 indicates a left bundle branch block (LBBB). In lead III, multiple peaked QRS complexes may show localized intraventricular conduction delays. Additionally, a QRS complex wider than 0.12 seconds without the specific characteristics of LBBB or RBBB points to a generalized intraventricular conduction delay (IVCD). Hypertrophy within the heart can be detected by assessing certain ECG features. Left Ventricular Hypertrophy (LVH) is characterized by tall R waves in leads I and V5-V6, coupled with deep S waves in V1-V2. A sum greater than 35 mm of the S wave depth in V2 and the R wave height in V5 is indicative of LVH. Right Ventricular Hypertrophy (RVH) is suggested by increased R wave amplitude in V1 and a deep S wave in V6, with an R:S ratio greater than 1 in V1. Atrial enlargement is also identifiable; left atrial enlargement shows as a broad and notched P wave in lead II (P-mitrale), while right atrial enlargement presents as a tall and peaked P wave in the same lead (P-pulmonale). (Only when there is no other diagnosable arrhythmia.) STTC is only significant when there is no other arrhythmia types, otherwise, abnormalities are diagnostic rather than STTC. Analyzing ST/T changes on an ECG requires a lookout for any obvious abnormalities. For example, wide and bizarre complexes interspersed with normal cycles may hint at ventricular tachycardia. ST depression and T waves that deviate from the normal direction are indicative of ischemia. ST elevation, which may or may not be accompanied by changes in T waves, denotes injury. The presence of pathological Q waves, especially in leads other than V1, is significant. Moreover, symmetrical or asymmetrical changes in the ST segment and T waves provide insights into the underlying pathology. Clear signs of ischemia or infarction are regional T wave abnormalities, ST segment changes, and the appearance of Q waves.

### Extracted (or Engineered) Features:

General: N/A Leads: —I: N/A, —II: N/A, —III: Notched R Wave. —aVF: N/A, —aVR: N/A, —aVL: N/A, —V1: N/A, —V2: N/A, —V3: N/A, —V4: N/A, —V5: N/A, —V6: N/A

### Augmenting Information:

Supplemental information on the ECG features: The notched R wave in lead III is not a cause for concern. It is just a part of the complex and does not indicate any abnormality.

Formatting Instructions: Based on the above guidance and considering the supplemental information, please categorize the ECG into the following arrhythmia categories: —Normal ECG (NORM) —ST/T Change (STTC) —Myocardial Infarction (MI) —Conduction Disturbance (CD) —Hypertrophy (HYP). Please output in a Json format of NORM:, MI:, STTC:, CD:, HYP: with the explanation of each type. For example: {NORM: {Result: True, Explanation: . . . }} Note: please directly provide the results in a JSON format with no other explanation.

### Response:

NORM: —Result: true, —Explanation: “The only feature detected is a notched R wave in lead III, which, according to the supplemental information, is not indicative of any abnormality.”
STTC: Result: false, Explanation: “There are no ST depressions, T wave inversions, or any other ST/T changes mentioned in the ECG features.”
MI: —Result: false, —Explanation: “There is no evidence of ST segment elevation, pathological Q waves, or reciprocal changes in any of the leads provided.”

### Example 2: ECG with Both CD and MI in PTBXL+

The prompt preface, diagnostic guidance, and formatting instructions are the same as in Example 1.

Features: The following information describe a 12-lead ECG in features.
General: Long PR interval of 214.0 ms, which might indicate a conduction disturbance or ST/T change. Prolonged QT interval detected with a corrected QT of 469.0 ms.
Leads: —I: Inverted T wave is approximated at −1.31 mm. Inverted T wave detected, which might suggest ST/T change if no other arrhythmia diagnosed. —II: Abnormal RS complex might indicate left anterior fascicular block (LAFB) as condution disturbance. —III: ST segment elevation is approximated at 1.26 mm. Abnormal RS complex might indicate Left anterior fascicular block (LAFB) as condution disturbance. —aVF: Abnormal RS complex might indicate Left anterior fascicular block (LAFB) as condution disturbance. —aVR: N/A —aVL: Inverted T wave is approximated at −2.14 mm. Prolonged R peak time, might indicate condution disturbance. Inverted T wave detected, which might suggest ST/T change if no other arrhythmia diagnosed. —V1: ST segment elevation is approximated at 2.19 mm. Deeper Q wave is approximated at amplitude of −22.99 mm. Q wave can be seen in lead V1, which might indicate an abnormal pattern. —V2: ST segment elevation is approximated at 3.95 mm. Abnormal T wave detected, which might suggest ST/T change if no other arrhythmia diagnosed. —V3: ST segment elevation is approximated at 2.63 mm. —V4: N/A —V5: N/A —V6: N/A

### Augmenting Information:

Supplemental information on the ECG features: Based on the information provided, the ECG shows a long PR interval, which may indicate a conduction disturbance. The ECG also shows ST segment elevation in leads III, aVF, V1, V2, and V3, which may suggest ST/T changes. Inverted T waves are seen in leads I and aVL, which may also indicate ST/T changes. Abnormal RS complexes in leads II, III, and aVF may suggest left anterior fascicular block as a conduction disturbance. The ECG also shows a prolonged QT interval. Overall, the ECG findings suggest possible conduction disturbances, hypertrophy, and ST/T changes.

### Response:

NORM: —Result: false, —Explanation: “The ECG shows significant abnormalities including ST segment elevation, inverted T waves, and abnormal RS complexes, indicating the presence of arrhythmias and excluding a normal ECG.”
STTC: Result: false, Explanation: “Diagnosable change in ST and T waves has been detected.”
CD: —Result: true, —Explanation: “There is no evidence of ST segment elevation, pathological Q waves, or reciprocal changes in any of the leads provided.”
MI: —Result: true, —Explanation: “Abnormal RS complexes in leads II, III, and aVF suggest a left anterior fascicular block. Prolonged PR and QT intervals also indicate conduction disturbances.”
HYP: —Result: false, —Explanation: “There is no direct evidence of hypertrophy such as tall R waves in leads V5-V6 or deep S waves in V1-V2 for LVH, or increased R wave amplitude in V1 and deep S wave in V6 for RVH.”

What is claimed:

1. A method for diagnosing a health condition, the method comprising:
obtaining observed electrocardiogram (ECG) data from an ECG machine;
extracting a plurality of features from the observed ECG data resulting in raw extracted ECG features;
modifying the raw extracted ECG features, using retrieval augmentation and according to a diagnosis guidance obtained from a database of domain knowledge, resulting in engineered ECG features;
obtaining augmentation information from the database of domain knowledge using, as a query, the engineered ECG features;
preparing a prompt comprising the engineered ECG features, the diagnosis guidance, and the augmentation information; and
determining a health condition diagnosis based on the prompt using zero-shot inference with a large language model (LLM).

2. The method of claim 1:
wherein preparing the prompt comprises categorizing the engineered ECG features resulting in categorized ECG features;
wherein the prompt comprises at least some of the categorized ECG features.

3. The method of claim 2:
wherein the engineered features are categorized into general ECG information and lead-wise ECG information;
wherein the lead-wise ECG information comprises engineered ECG features specific to one or more leads of the engineered ECG data.

4. The method of claim 3, wherein the prompt further comprises formatting instructions relating to a format of the health condition.

5. The method of claim 1, wherein the diagnosis guidance is obtained by determining a subset of the raw extracted ECG features that are optimal for diagnosing a preselected health condition.

6. The method of claim 5, wherein the engineered ECG features comprise the subset of the raw extracted ECG features determined by the diagnosis guidance.

7. The method of claim 1, wherein extracting the plurality of features from the observed ECG data comprises pretraining an ECG data encoder and using the pretrained ECG data encoder to extract the plurality of features from the observed ECG data.

8. A system for diagnosing a health condition, the system comprising:
an electrocardiogram (ECG) machine;
a database of domain knowledge relating to ECGs; and
a computer communicatively coupled to the ECG machine and configured to:
receive observed ECG data from the ECG machine,
extract a plurality of features from the observed ECG data resulting in raw extracted ECG features,
modify the raw extracted ECG features, using retrieval augmentation and according to a diagnosis guidance obtained from the database of domain knowledge, resulting in engineered ECG features,
obtain augmentation information from the database of domain knowledge using, as a query, the engineered ECG features;
prepare a prompt comprising the engineered ECG features, the diagnosis guidance, and the augmentation information, and
determine a health condition diagnosis based on the prompt using zero-shot inference with a large language model (LLM).

9. The system of claim 8:
wherein preparing the prompt comprises categorizing the engineered ECG features resulting in categorized ECG features;
wherein the prompt comprises at least some of the categorized ECG features.

10. The system of claim 9:
wherein the engineered features are categorized into general ECG information and lead-wise ECG information;
wherein the lead-wise ECG information comprises engineered ECG features specific to one or more leads of the engineered ECG data.

11. The system of claim 8, wherein the diagnosis guidance is obtained by determining a subset of the raw extracted ECG features that are optimal for diagnosing a preselected health condition.

12. The system of claim 11, wherein the engineered ECG features comprise the subset of the raw extracted ECG features determined by the diagnosis guidance.

13. The system of claim 8, wherein extracting the plurality of features from the observed ECG data comprises pretraining an ECG data encoder and using the pretrained ECG data encoder to extract the plurality of features from the observed ECG data.

14. A non-transitory computer-readable medium storing instructions that, when executed by a processor, cause the processor to perform a method comprising:
obtaining observed electrocardiogram (ECG) data from an ECG machine;
extracting a plurality of features from the observed ECG data resulting in raw extracted ECG features;
modifying the raw extracted ECG features, using retrieval augmentation and according to a diagnosis guidance obtained from a database of domain knowledge, resulting in engineered ECG features;
obtaining augmentation information from the database of domain knowledge using, as a query, the engineered ECG features;
preparing a prompt comprising the engineered ECG features, the diagnosis guidance, and the augmentation information; and
determining a health condition diagnosis based on the prompt using zero-shot inference with a large language model (LLM).

15. The non-transitory computer-readable medium of claim 14:
wherein preparing the prompt comprises categorizing the engineered ECG features resulting in categorized ECG features;
wherein the prompt comprises at least some of the categorized ECG features.

16. The non-transitory computer-readable medium of claim 15:
wherein the engineered features are categorized into general ECG information and lead-wise ECG information;
wherein the lead-wise ECG information comprises engineered ECG features specific to one or more leads of the engineered ECG data.

17. The non-transitory computer-readable medium of claim 16, wherein preparing the prompt further comprises formatting instructions relating to a format of the health condition.

18. The non-transitory computer-readable medium of claim 14, wherein the diagnosis guidance is obtained by determining a subset of the raw extracted ECG features that are optimal for diagnosing a preselected health condition.

19. The non-transitory computer-readable medium of claim 18, wherein the engineered ECG features comprise the subset of the raw extracted ECG features determined by the diagnosis guidance.

20. The non-transitory computer-readable medium of claim 14, wherein extracting the plurality of features from the observed ECG data comprises pretraining an ECG data encoder and using the pretrained ECG data encoder to extract the plurality of features from the observed ECG data.
